(19) United States
(12) Patent Application Publication
(10) Pub. No.: US 2009/0048650 A1
(43) Pub. Date: Feb. 19, 2009

(54) CONFIGURABLE ICE PACK AND HEATING PAD

(76) Inventor: Mitch Junkins, Newport Beach, CA (US)

Correspondence Address: Rutan & Tucker, LLP., 611 ANTON BLVD, SUITE 1400, COSTA MESA, CA, 92626, US

(21) Appl. No.: 12/191,649

(22) Filed: Aug. 14, 2008

Related U.S. Application Data
(60) Provisional application No. 60/955,818, filed on Aug. 14, 2007.

(51) Int. Cl. A61F 7/08; A61F 7/10

(52) U.S. Cl. 607/112; 607/114

(57) ABSTRACT

An apparatus and a system is provided for an improved apparatus for the alleviation of minor pain. The new and unique apparatus for may be utilized for use in the treatment of minor aches and pains whereby the apparatus may be configurable and may conform to a specific pain emanating region of the body, yet have enough tension through the design to hold the device to a specific painful area of the body. Moreover, the apparatus may utilize a plurality of different filler material thereby allowing for use as either a heat based or cooling based device depending on the use and desires of the individual user and/or the type of pain to be alleviated by the user.

### FIELD OF THE INVENTION

The field of the invention is for a ice pack and/or heating pad. More specifically, the field of invention is for a configurable, adaptable ice pack and/or heating device capable of conforming to a specific patient location.

### BACKGROUND

Pain management has become an increasingly serious concern for many people. For this reason, with the increase in age, stress and physical activities, many individuals endure various levels of aches and pain. Management of this pain can range from medication to herbal solutions. However, for those individuals that do not want medication for temporary soreness, aches and pains, there are fewer solutions which require inactivity and substantial immobility.

One common type of device utilized to treat aches and pains is the heating pad. The heating pad is a pad that may be utilized to manage pain. The heating pad may be applied to a localized region of an individual's body to combat the pains. The heat typically will cause the blood vessels in a particular part of the body to dilate, which will allow for greater blood flow and perfusion of needed relaxation of the muscles associated with the pain. Typical, heating pads are electrical which requires some electrical power source such as a battery or wall outlet. Additionally, these electrical heating pads tend to be bulky and hard to move from location to other locations. Additionally, the shape of the heating pads are typically dictated by the electrical sources of power, because of the need to keep the entire pad warmed, electrical wiring or other components are located throughout the pad. Because of these electrical components, it is often necessary for the user to maintain their position in a substantially stationary position while they allow the warming effects to work.

Another type of heating pad is the heating bottle. The heating bottle is warmed by placing the device into a warming facilitator such as a microwave oven. After warming, the pad is typically placed on the localized pain area for application of heat thereto. These type of heating bottles or pads, are typically made of thick, isolative fabric such as flannel and filled with any heat absorbent material such as flax seed or grain. However, because of the external shape of the pad and the need to keep the contents of the pad in the interior portion, the outside portions of the pad are typically restrictive and hard to conform to a particular region of the body without constructing the pad into a customized shape to fit a specific location of the body.

Another prior art pain management source is the common ice pack. The ice pack is typically utilized to alleviate the pain of minor injuries. The ice pack tends to stop the swelling of joints and other muscular pain. Most typical ice packs are made of an outside plastic coating and are filled with some type of refrigerant that may retain cooling properties for a duration of time.

However, a significant problem with common ice packs are their outside coating tends to be made of highly resilient and inflexible materials such as hard plastic to keep the refrigerant in the interior portion of the pack.

Another problem that exists, is that these ice packs are typically very small and cannot cover an extensive portion of the desired pain area.

Therefore, a need exists for an improved pain alleviation pad that may be configurable and conformable about the desired area of the user.

Additionally, a need therefore exists for an improved pain alleviation pad that may have a tension portion which may hold it to the sore/painful site desired by the individual. Additionally, a need exists for an improved pain alleviation pad that may utilize a plurality of different internal materials that may either cool and/or heat the pains of the individual depending on the desired choice of that individual that intends to alleviate minor pain.

### SUMMARY OF THE INVENTION

The present invention provides an apparatus and a system for an improved apparatus for the alleviation of minor pain. Additionally, the present invention provides an improved apparatus for use in the treatment of minor pain, whereby the apparatus may conform to a specific painful area of the body, yet have enough tension through the design to hold the device into a specific painful area of the body. Moreover, the present invention may utilize a plurality of different filler material thereby allowing for use as either a heat based or cooling based device depending on the use and desires of the individual user and/or the type of pain to be alleviated by the user. Additionally, the apparatus and system may have an opening also having a locking mechanism to allow for multiple use of different types of fillers of the apparatus for use by the individual whereby the user may use the apparatus for heating and/or cooling of a minor pain.

To this end, in an exemplary embodiment of the present invention, an apparatus for use in the alleviation of minor pain is provided. The apparatus has at least an outside portion and an interior portion containing an interior space which may be filled with a filler material. The apparatus has at least a filler portion having at least an opening thereon for insertion and removal of the filler material. Further, the apparatus has a locking mechanism configured to seal the interior portion of the apparatus.

In an exemplary embodiment, the apparatus has an outside portion that is configured to allow for conformation and tension to the desired site of pain.

In an exemplary embodiment, the apparatus has an outside portion that is configured to allow for substantial affixation to the desired site of pain while still retaining a rigid configuration.

In an exemplary embodiment, the apparatus has an outside portion that is constructed of a suitable material whereby the material is a flexible silicon rubber.

In an exemplary embodiment, the apparatus has an interior portion that is filled with a refrigerant gel.

In an exemplary embodiment, the apparatus has an interior portion that is filled with rice.

In an exemplary embodiment, the apparatus has an interior portion that is filled with beans, grains or other filler material.

In an exemplary embodiment, the apparatus has an interior portion that may be filled and emptied of its contents to allow the apparatus to be utilized for both hot and cool operation by the user.

In an exemplary embodiment, the apparatus has tensioning abilities whereby the apparatus may allow for increase mobility of the individual user when the apparatus is in use based on the apparatus ability to cling to the desired portion of the body in need of alleviation of minor pain.

In an exemplary embodiment, the apparatus has a locking mechanism containing a temperature indicator means.

In an exemplary embodiment, the apparatus has a locking mechanism that contains a threadable portion adapted to thread into a similarly configured threaded portion on the filler portion of the apparatus to seal the interior portion of the apparatus.

In an exemplary embodiment, the apparatus has a plurality of conduits on the apparatus to allow for continual heating of the filler material in the interior portion of the apparatus.

In an exemplary embodiment, the apparatus has at least a conduit on the apparatus to allow for substantially continual cooling of the filler material contained in the interior portion of the apparatus.

In an exemplary embodiment, the apparatus has a power source to power an indicator means whereby the indicator means comprises a temperature indicator, and an audio/visual indicator.

To this end, in an exemplary embodiment of the present invention, a method of utilizing a pain relieving apparatus, the method comprising the steps of: providing an apparatus having at least an outside portion; an interior portion containing an interior space; allowing the insertion of a filler material into the interior portion of the apparatus; providing a filler portion having an opening thereon for insertion and removal of the filler material; and providing a locking mechanism configured to seal the interior portion of the apparatus.

In an exemplary embodiment a method for utilizing a pain relieving apparatus further comprises the step of: allowing a user to fill the interior portion of the apparatus with a filler material wherein the filler material comprising any of: ice, water, rice, beans, grains, refrigerant gel and other fillers.

In an exemplary embodiment a method for utilizing a pain relieving apparatus further comprises the step of: providing an indicator means on the apparatus to illustrate the temperature of the interior portion of the apparatus to the individual user.

In an exemplary embodiment a method for utilizing a pain relieving apparatus further comprises the step of: allowing for sealing of the interior portion of the apparatus by applying a locking mechanism to the opening of the filler portion.

In an exemplary embodiment a method for utilizing a pain relieving apparatus further comprises the step of: applying the apparatus to the desired portion of the body and configuring the apparatus to allow for substantial affixation to the desired site of pain while still retaining a rigid configuration.

In an exemplary embodiment a method for utilizing a pain relieving apparatus further comprises the step of: allowing for removal of the filler material from the interior portion of the apparatus to allow for refilling of the interior portion with a different filler material.

Among the many different possibilities contemplated, the apparatus may allow for multiple configurations to hold specific body parts.

Additionally, in an exemplary embodiment, the apparatus may be configured to accommodate a plurality of different body parts including the neck of an individual, the knee, elbow and the like.

In another exemplary embodiment, it is contemplated that the apparatus may be configurable to hold onto a sore muscle group.

In yet another exemplary embodiment, it is contemplated that the apparatus may have a tension portion whereby the tension portion may allow for grasping and substantial embracement of the apparatus to a particular muscle group and/or portion of the body.

In still another exemplary embodiment, it is contemplated that the apparatus be constructed of a suitable material which may provide adequate tensioning such that the apparatus may be able to substantially maintain its position about a particular part of the anatomy.

A further exemplary embodiment contemplates that the apparatus may be constructed of a suitable material such as plastic.

Yet another exemplary embodiment contemplates that the apparatus may be constructed of rubber, a polyester, or any other suitable material that will provide the necessary tensioning, yet at the same time provide adequate flexibility to allow for conforming of the apparatus to a particular part of the anatomy.

It is further contemplated that the apparatus may be constructed to be adaptable to a plurality of body parts of the human anatomy, however, it should be understood that the apparatus may be configured to be adaptable and configurable to fit the anatomy of different animals, including household pets, horses, zoo animals and the like.

Further, a contemplated embodiment of the apparatus may have an outer portion and an interior portion whereby the outer portion and the interior portion may be constructed of different materials.

Yet another exemplary embodiment is to provide an apparatus for minor pain relief whereby the apparatus may be constructed of a suitable tensioning material and further wherein the apparatus may have an interior portion which may be filled with a completely different substance.

Still another exemplary embodiment is to provide an apparatus for minor pain relief whereby the apparatus may be constructed of a suitable tensioning material and further wherein the apparatus may have an interior portion which may be filled with a material and subsequently emptied by the user and re-filled with a completely different filler material.

Additionally, in an exemplary embodiment, the apparatus may have a outer portion whereby the outer portion may be constructed of plastic.

In an exemplary embodiment, the apparatus may have an exterior portion which may be constructed of a flexible silicone rubber.

A further exemplary embodiment of the present invention may include an apparatus for alleviation of minor pain whereby the apparatus may have an outer portion made of any suitably rigid material that may have dexterity to move and conform to the body location of an individual while still having at least a flexibility and a tensioning characteristic.

Still another exemplary embodiment of the present invention may include an apparatus having an outer portion and an interior portion whereby the interior portion may be filled by a suitable material by the individual user.

In yet another exemplary embodiment of the present invention, an apparatus to be used for minor pain relief may be provided whereby the apparatus may fill the apparatus with a plurality of different materials including: ice, rice, beans, gel, water, or any other filler materials which may be utilized to heat and/or cool the entire apparatus.

A further exemplary embodiment of the present invention may include an apparatus whereby the apparatus may be utilized to cool a particular region of the user's anatomy and/or may be utilized to heat a particular region of the user's anatomy.

In yet another exemplary embodiment of the present invention, an apparatus may be provided whereby the apparatus may be utilized interchangeably for heating and cooling of a particular aches and/or pain.

Yet another exemplary embodiment of the present invention may include an apparatus having an outer portion and an interior portion whereby the interior portion may be filled with rice.

In an exemplary embodiment of the present invention, an apparatus may be provided whereby the apparatus may have an outer portion and an interior portion whereby the interior portion may be filled with beans.

Another exemplary embodiment of the present invention may include an apparatus to alleviate minor pain whereby the apparatus may have an outer portion and an interior portion whereby the interior portion may be filled with ice.

In still another exemplary embodiment of the present invention an apparatus to alleviate minor pain is provided whereby the apparatus may have an outer portion and an interior portion whereby the interior portion may be filled with a refrigerant type gel.

In an exemplary embodiment of the present invention, an apparatus for alleviation of minor pain may be provided whereby the apparatus may be utilized with any cooling agent.

Still, in another exemplary embodiment of the present invention, an apparatus for alleviation of minor pain may be provided whereby the apparatus may be utilized with any heating agent.

In yet another exemplary embodiment of the present invention, an apparatus for alleviation of minor pain may be provided whereby the apparatus may have a specially featured design allowing for adhesion to a particular muscle or body part.

Further, in another exemplary embodiment of the present invention, an apparatus for alleviation of minor pain may be provided whereby the apparatus may have a special exterior contour allowing for proper tension onto a sore muscle group.

In another exemplary embodiment of the present invention, an apparatus for alleviation of minor pain may be provided whereby the apparatus may have an adhesive portion thereon to allow for proper grasping and holding of the muscle group thereby allowing for increase mobility of the individual utilizing the apparatus.

Another exemplary embodiment of the present invention is to provide an apparatus for alleviation of minor pain whereby the apparatus may have a filling neck located on a portion of the apparatus whereby filler material may be inserted and removed from the filling neck.

Yet another exemplary embodiment of the present invention is to provide an apparatus for alleviation of minor pain whereby the apparatus may have a filling neck defined by an opening thereon whereby the opening may be closed and locked with the inclusion of a locking mechanism which may seal the interior portion of the apparatus with whatever material has be inserted therein.

Still another exemplary embodiment of the present invention is to provide an apparatus for alleviation of minor pain whereby the apparatus may have a filling neck having an opening whereby a locking mechanism with locking indicators may be provided to allow the user to lock the apparatus and know when the apparatus has been properly sealed.

In yet another exemplary embodiment of the present invention, an apparatus for alleviation of minor pain may be provided whereby the apparatus may have a locking cap whereby the locking cap may have a thermometer located thereon to indicate to the individual user the temperature of the contents of the apparatus and/or of the entire apparatus itself.

Still a further exemplary embodiment of the present invention is to provide an apparatus for alleviation of minor pain whereby the apparatus may allow for increased mobility of the individual over the mobility afforded by prior art heating/cooling apparatus.

Various objects, features, aspects and advantages of the present invention will become more apparent from the following detailed description of preferred embodiments of the invention, along with the accompanying drawings in which like numerals represent like components.

### BRIEF DESCRIPTION OF THE DRAWING

FIG. 1 is a front perspective view of the invention illustrating the apparatus in an exemplary embodiment of the present invention;

FIG. 2 is a side perspective view of the invention showing the side of the apparatus in an exemplary embodiment of the present invention;

FIG. 3 is a cross sectional view of the apparatus in an exemplary embodiment of the present invention;

FIG. 4 is a top view of the apparatus in an exemplary embodiment of the present invention; and

FIG. 5 is a cross-sectional perspective view of the invention illustrating the apparatus in an exemplary embodiment of the present invention.

### DETAILED DESCRIPTION OF THE PREFERRED EMBODIMENTS

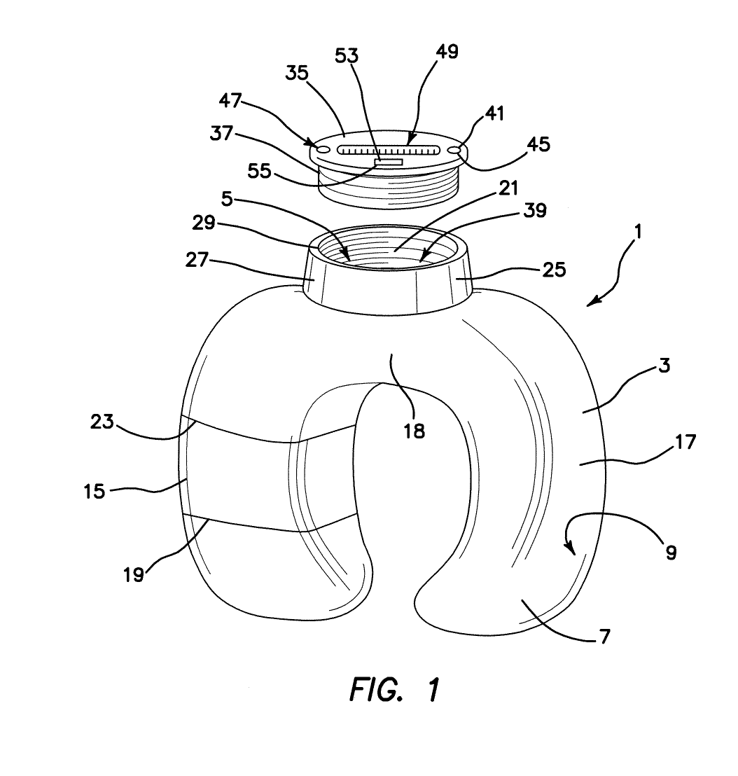

Turning now to the drawings where the apparatus is illustrated in the five Figures, FIG. 1 illustrates a front perspective of the apparatus 1 having a contoured shaped. In an exemplary embodiment, the apparatus 1 may have at least an exterior portion 3 and an interior portion 5. The exterior portion 3 may contact a body part of an individual (not shown). The interior portion 5 may be defined by a void that may be filled by a suitable material. The exterior portion 3 may have an outside edge 7 and an inside edge 9. It is contemplated that the exterior portion 3 may be made of a singular material such as a polyester, plastic of in a preferred embodiment, a flexible silicone rubber. However, it should be understood that any suitable material may be utilized in the construction of the apparatus 1 as long as said material is capable of flexing about the body part of an individual and further wherein the material should be capable of tensioning about a particular part of the anatomy of the user.

The exterior portion 3 may also have an inside edge 9 and an outside edge 7 whereby it is contemplated that the inside edge 9 and the outside edge 7 may be constructed of a the same material. However, it is also contemplated that the inside edge 9 of the apparatus 1 may be constructed of a different material than the outside edge 7. The advantage to the use of a different material may be that the inside edge 9 may better hold a filler material 59 if constructed of a particular material. Further, it the outside edge 7 may be adapted to be constructed of a material having elastomeric properties which may better conform to the particular angles and configurations of the individual to which fitment is desired. Additionally, the use of pliable and more comfortable materials on the outside edge 7 of the apparatus 1 may allow for easier use and more comfort to the user.

FIG. 1 further illustrates the apparatus having a contoured shape. In an exemplary embodiment, the apparatus 1 may have a first finger 15 and a second finger 17. The first finger 15 and second 17 may be of identical lengths and sizes. However, it should be contemplated that the first finger 15 and second protrusion 17 may have different lengths and sizes to accommodate a plurality of different body parts as desired by the individual user. Additionally, the first and second fingers 15, 17 may be further retractable if desired such that the first finger 15 may retract toward a midpoint 18 of the apparatus such that the first finger 15 may be shorter in length than the second finger 17. The fingers 15, 17 may have a retraction mechanism 19 located thereon which may allow the finger to retract into an interior space 21 of the apparatus 1. It is further contemplated that the fingers 15, 17 may have at least a first retraction mechanism 19 and in an exemplary embodiment, may have a second retraction mechanism 23. Because the apparatus 1 and more specifically, the outside edge 7 of the apparatus 1 may be constructed of a flexible material such as a flexible silicone rubber, it may be constructed to be pliable enough to retract inside another portion of the finger to reduce the overall length of the first finger 15 or second finger 17 or both.

Further illustrated in FIG. 1, the apparatus 1 may have a first finger 15 and a second finger 17. However, it should be understood that the apparatus 1 may include a plurality of fingers or just one finger 1. In an exemplary embodiment, the fingers 15 and 17 may produce what may look like a horse shoe which may allow for easier configuration of the apparatus around certain body parts including the elbow, knees and shoulders which are particularly difficult to apply heating and cooling to without great difficulty.

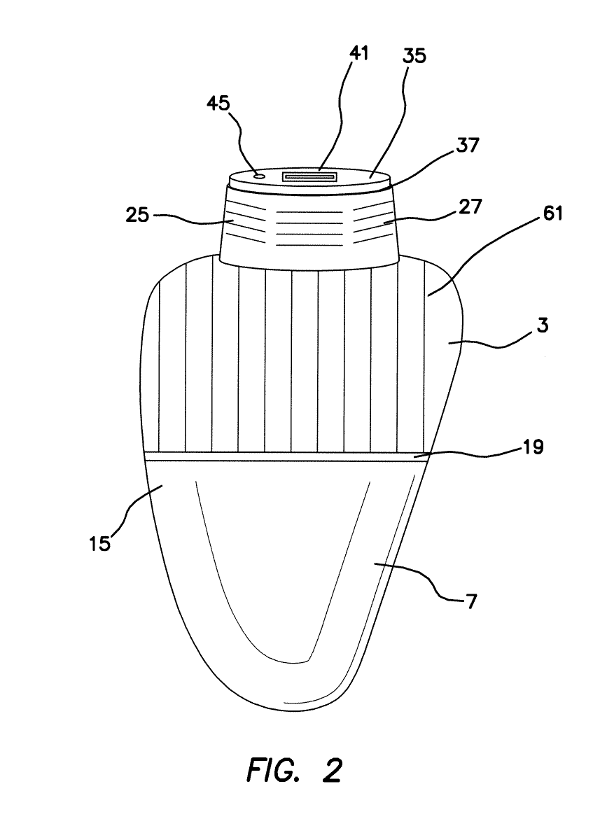

FIGS. 1 and 2 further illustrated the top filler portion 25. The filler portion 25 may have a protrusion 27 which extends away from the outside edge 7 of the apparatus 1. The protrusion 27 may extend away from the outside edge 7 and further may be defined by an opening 29 which may encompass the majority of the filler portion 25. The opening may have a threaded portion 31 which may be configured to accommodate a similarly threaded portion on a locking mechanism 35. The locking mechanism 35 may have a threaded portion 37 which may be accommodated by the threaded portion 31 of the filler 25. The locking mechanism 35, in an exemplary embodiment, may be a cap which may seal the interior space of the apparatus when it is properly inserted into and about the opening 29 of the filler portion 25.

Further illustrated on both FIGS. 1 and 2, the locking mechanism 35 may be inserted onto the filler portion 25 to secure the contents of the apparatus 1. The locking mechanism may be secured by using a threaded mechanism which seals the interior space 39 of the apparatus 1. However, any suitable locking mechanism may be utilized such as straps, clips, snaps, and the like. However, because the apparatus 1 may utilize liquid substances in the interior space 39, it is advantageous for the locking mechanism 35 to provide a tight sealed connection with the apparatus 1 such that the contents of the apparatus may not escape from the interior space 39 of the apparatus 1 when the device is in use.

Further, it is contemplated that the locking mechanism 35 may a temperature indicator 41 located on the locking mechanism 35 which may allow the user to know the temperature of the contents of the interior space 39 of the apparatus 1. By utilizing the temperature indicator 41, the user will know the relative temperature of the contents such that the individual may heat or cool the apparatus 1 more thoroughly when desired. Further, the temperature indicator 41 may inform the user that the apparatus 1 may be too hot or too cold for contact with a body part of the individual user. Further, the temperature indicator 41 may also indicate the temperature of the actual apparatus 1 whereby knowing the temperature of the device will allow the individual user to know when it is appropriate or when the apparatus 1 is ready for use. It is contemplated that the temperature indicator 41 may also be located anywhere on the apparatus 1, but preferably be located on the locking mechanism 35 because of the protrusive nature of its location. In an exemplary embodiment an audio/visual indicator 45 may be provided which may inform the use when the apparatus 1 is ready for use. The audio/visual indicator 45 may be pre-programmed to indicate when a set temperature is reached and to then notify the user that the apparatus 1 is ready for use. The audio/visual indicator 45 may be in the form of a simple light 47 or a audible speaker 49 which sends a simple audio single that the apparatus 1 is ready for use upon reaching the pre-determined temperature setting. The temperature indicator 41 may be a thermometer 49 which will show the temperature of the interior space 39 of the apparatus 1. The thermometer 49 may be of a mechanical type which utilizes mercury therein, or in an exemplary embodiment, may be a digital type. If the temperature indicator 41 utilizes digital readouts and/or audio/visual indicators 45, a power source 53 may be supplied. The power source 53 may be a battery 55 which may power the audio/visual indicator 45 and the temperature indicator 41. The battery 55 may be contained within the locking mechanism 35 or may be located anywhere within the apparatus 1 itself.

An additional feature of the present invention, is to utilize the battery 55 or power source 53 throughout the apparatus 1 whereby the power source 53 may be utilized to continually heat or cool the contents of the interior space 39 of the apparatus 1 after the filler material 59 has been inserted into the apparatus 1. In this way, the individual does not have to continually heat up or cool the apparatus 1 when heat dissipation or cooler power has receded. The power source 53 may be used to conduct heat throughout a plurality of conduits 61 in the exterior portion 3 of the apparatus 1. The conduits 61 may keep the filler material 59 heated if desired by the individual for a longer period of time. Additionally, the conduits 61 may be utilized to continually keep the filler materials 59 cool when utilized.

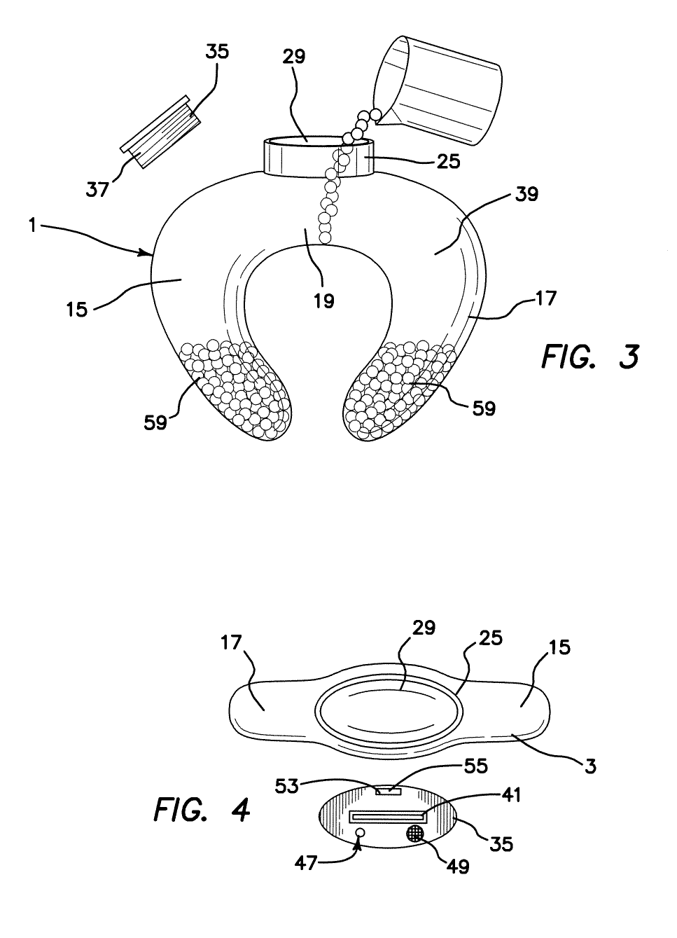

FIG. 3 illustrates a cross sectional view of the apparatus 1 in an exemplary embodiment. As illustrated, a filler portion 25 may be provided having an opening 29. An individual may utilized the opening 29 to insert a filler material 59. The filler material 59 may be inserted into the filler portion 25 through the opening 29 when the locking mechanism 35 is removed therefrom. A filler material 59 may be a liquid such as water, or a solid such as beans or rice. Additionally, the filler material 59 may be a gel which may be heated or cooled by the individual user after insertion into the interior space 39 of the apparatus 1. In an exemplary embodiment, the filler material 59 may be water, ice, beans, rice, gel, or any other material that is capable of being cooled or heated to be inserted into the interior space 39 of the apparatus 1.

As can be seen in FIG. 3, any amount of filler material 59 may be inserted into the interior space 39 of the apparatus 1. The advantage of allowing the user to fill as much of the interior space 39 of the apparatus may be to give the right amount of heating or cooling area which may be desired by the individual user. The interior space 39 may be completely filled or only partially filled by the individual user. Additionally, the interior space 39 may be easily emptied of its filler material 59 content and replaced with another filler material 59 as desired by the individual. In this way, the user may refuse the apparatus for a plurality of different pain relief functions including both hot and cold treatment of minor pain.

FIG. 4 illustrates a top view of the apparatus 1 in an exemplary embodiment of the present invention. As can be seen, the filler portion 25 is illustrated showing the opening 29 whereby an individual user may utilize the filler material 59 and insert same into the opening 29 to fill a portion of the interior space 39 of the apparatus 1. The locking mechanism 35 is also illustrated in FIG. 4 showing the temperature indicator 41 in the form of a thermometer 49. Additionally, the audio/visual indicator 45 is also illustrated in the form of both an light source 47 and speaker 49. Additionally, the power supply 53 in the form of a battery 55 is also illustrated as previously enumerated.

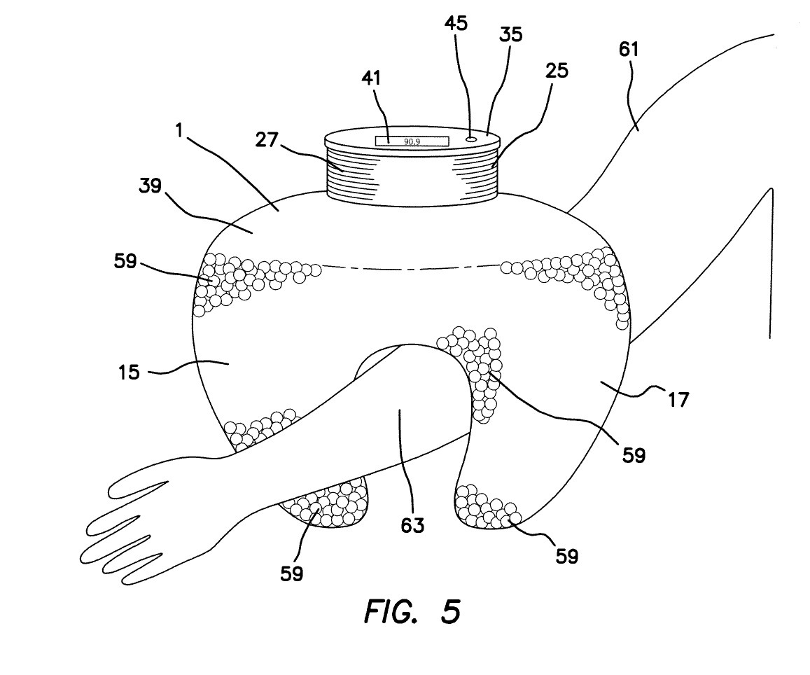

FIG. 5 illustrates the user's 61 arm 63 utilizing the apparatus 1. As can be seen, the user 61 has inserted the filler material 59 which in this exemplary embodiment may be rice 65. The entire apparatus 1 may be heated in a microwave or other heating source and then may be utilized to heat a particular portion of the user's 61 body, in this case the arm 63. The construction of the apparatus 1 may allow for the apparatus 1 to conform to the individual body part, yet have enough tension through the design of the apparatus 1 and the construction material to grasp and substantially hold the muscle or body part to be utilized by the apparatus 1. FIG. 5 also illustrates the locking mechanism 35 in its closed position whereby the interior space 39 of the apparatus 1 is completely sealed from leakage of the filler material 59 from the interior space 39. Additionally, the individual user 61 may be able to see the temperature indicator 41 to determine if the right amount of heat or cold is being applied. The temperature indicator 41 may also be beneficial in the sense that often times, the sensory perception of an individual user 61 may be inaccurate after the application of heating and or cooling of that body part for a period of time. In this way, the temperature indicator 41 may be able to illustrate more concisely to the individual user 61 that the right amount of heating and/or cooling is being properly applied.

Thus, specific embodiments and applications of wall mounting bracket installer with attachable wing systems and methods have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

I claim:

1. An apparatus for use in the alleviation of minor pain, the apparatus comprising:
at least an outside portion;
an interior portion containing an interior space which may be filled with a filler material;
at least a filler portion having at least an opening thereon for insertion and removal of the filler material; and
a locking mechanism configured to seal the interior portion of the apparatus.

2. The apparatus described in claim 1 wherein said outside portion is configured to allow for conformation and tension to the desired site of pain.

3. The apparatus described in claim 1 wherein said outside portion is configured to allow for substantial affixation to the desired site of pain while still retaining a rigid configuration.

4. The apparatus described in claim 1 wherein said outside portion is constructed of a suitable material whereby the material is a flexible silicon rubber.

5. The apparatus described in claim 1 wherein said interior portion is filled with a refrigerant gel.

6. The apparatus described in claim 1 wherein said interior portion is filled with rice.

7. The apparatus described in claim 1 wherein said interior portion is filled with beans, grains or other filler material.

8. The apparatus described in claim 1 wherein the interior portion may be filled and emptied of its contents to allow the apparatus to be utilized for both hot and cool operation by the user.

9. The apparatus described in claim 1 wherein the apparatus may be tensioning abilities whereby the apparatus may allow for increase mobility of the individual user when the apparatus is in use based on the apparatus ability to cling to the desired portion of the body in need of alleviation of minor pain.

10. The apparatus described in claim 1 further comprising:
the locking mechanism containing a temperature indicator means.

11. The apparatus described in claim 1 wherein the locking mechanism contains a threadable portion adapted to thread into a similarly configured threaded portion on the filler portion of the apparatus to seal the interior portion of the apparatus.

12. The apparatus described in claim 1 further comprising:
a plurality of conduits on the apparatus to allow for continual heating of the filler material in the interior portion of the apparatus.

13. The apparatus described in claim 1 further comprising:
at least a conduit on the apparatus to allow for substantially continual cooling of the filler material contained in the interior portion of the apparatus.

14. The apparatus described in claim 1 further comprising:
a power source to power an indicator means whereby the indicator means comprises a temperature indicator, and an audio/visual indicator.

15. A method of utilizing a pain relieving apparatus, the method comprising the steps of:
providing an apparatus having at least an outside portion; an interior portion containing an interior space;
allowing the insertion of a filler material into the interior portion of the apparatus;
providing a filler portion having an opening thereon for insertion and removal of the filler material; and
providing a locking mechanism configured to seal the interior portion of the apparatus.

16. The method of claim 15 further comprising the step of:
allowing a user to fill the interior portion of the apparatus with a filler material wherein the filler material comprising any of: ice, water, rice, beans, grains, refrigerant gel and other fillers.

17. The method of claim 15 further comprising the step of:
providing an indicator means on the apparatus to illustrate the temperature of the interior portion of the apparatus to the individual user.

18. The method of claim 15 further comprising the step of:
allowing for sealing of the interior portion of the apparatus by applying a locking mechanism to the opening of the filler portion.

19. The method of claim 15 further comprising the step of:
applying the apparatus to the desired portion of the body and configuring the apparatus to allow for substantial affixation to the desired site of pain while still retaining a rigid configuration.

20. The method of claim 15 further comprising the step of:
allowing for removal of the filler material from the interior portion of the apparatus to allow for refilling of the interior portion with a different filler material.
